(19) United States
(12) Patent Application Publication
(10) Pub. No.: US 2003/0152957 A1
(43) Pub. Date: Aug. 14, 2003

(54) Probe-immobilized reaction arrays

(75) Inventors: Etsuo Shinohara, Hachioji-shi (JP); Takeo Takahashi, Tokyo (JP); Nobuhiko Morimoto, Hachioji-shi (JP)

Correspondence Address: Scully, Scott, Murphy & Presser, 400 Garden City Plaza, Garden City, NY, 11530, US

(73) Assignee: OLYMPUS OPTICAL CO., LTD., TOKYO (JP)

(21) Appl. No.: 10/215,062

(22) Filed: Aug. 8, 2002

(30) Foreign Application Priority Data
Feb. 12, 2002 (JP) 2002-034197

(51) Int. Cl. C12Q 1/68; G01N 33/53; C12M 1/34

(52) U.S. Cl. 435/6; 435/7.1; 435/287.2

(57) ABSTRACT

The present invention provides a probe-immobilized reaction array, comprising a substrate, first to nth reaction portions comprising bores formed independently in the inside of the substrate wherein n is an integer of 2 or more, two openings open from each of the first to nth reaction portions to a first face of the substrate, and a probe group consisting of a 1st probe group immobilized on at least one face of the first reaction portion to an nth probe group immobilized on at least one face of the nth reaction portion wherein n is an integer of 2 or more, wherein the first to nth probe groups comprise plural kinds of probes respectively, and the optimum reaction conditions of the probes are complete for each of the reaction portions.

### CROSS-REFERENCE TO RELATED APPLICATIONS

This application is based upon and claims the benefit of priority from the prior Japanese Patent Application No. 2002-034197, filed Feb. 12, 2002, the entire contents of which are incorporated herein by reference.

### BACKGROUND OF THE INVENTION

1. Field of the Invention

The invention relates to a reaction array to be used for analysis related to analysis of nucleic acids or proteins. In particular, the invention relates to a method of analyzing nucleic acids or proteins by utilizing specific binding.

2. Description of the Related Art

The basic principle of DNA microarrays is as follows. That is, a large number of different DNA probes are immobilized at high density onto a solid-phase carrier or substrate such as glass. A sample is added thereto, and a labeled target DNA contained in the sample is hybridized therewith. Thereafter, a signal derived from the label in each labeled target DNA thus hybridized is detected by an automatic detector, and the data thus obtained are analyzed in large quantity by a computer.

It is expected that such DNA microarrays will be able to achieve analysis of many kinds of nucleic acids in large quantity, improvement of detection sensitivity, reduction of the volume of a sample by microanalysis, automatic acquisition of data and simplification of data processing.

At present, DNA microarrays are divided roughly into two types depending on their difference in the production process. One type is also called DNA chips wherein DNA probes typically those from Affymetrix Ltd. are synthesized on a glass surface (Proc Natl Acad Sci USA 91:5022-5026 (1994)). The other type is also generally called DNA arrays wherein previously prepared DNA probes are mechanically arranged (Science 270:467-470 (1995)). At present, however, there is no accurate distinction between DNA microarray and DNA chip.

DNA microarrays wherein DNA probes have been spotted to a slide can be produced by a DNA-arraying machine. Such DNA microarrays are also advantageous in that DNA probes to be attached can be arbitrarily selected. On the other hand, a collection of DNA probes should be prepared. This is one cause making the production process troublesome. Attachment of DNA probes to such DNA arrays is conducted by physical spotting with the tip of a pin. Accordingly, this is inferior to the photolithographic system in high density of DNA probes. However, when spots of 100 μm in diameter are spotted at 100 μm intervals, 2500 spots can theoretically be arranged per 1 cm². Accordingly, when the effective area is assumed to be 4 cm², about 10,000 DNA probes can be spotted on one conventional slide.

The DNA arraying machine is a unit which is provided basically with a high-performance servomotor, wherein a pin tip or a slide holder is operated in the XYZ axial direction under the computer control, and a solution containing DNA probes is transferred from a microtiter plate to the surface of a slide. The shape of the pin tip is contrived thus constituting the life of this technology. The most general pin tip is a pin tip divided like a crow's bill. A DNA probe solution is stored therein and spotted onto a plurality of slides. Spotting of one DNA probe solution is followed by a cycle of washing and/or drying and subsequent spotting of another DNA probe solution. This process is repeated. The function required of the ideal DNA-arraying machine is that spots having uniform size and shape are produced rapidly with good reproducibility. In the conventional pin tip, there is a limit to uniformity and speed depending on a difference in lots. Accordingly, development of new techniques such as the ink jet system and capillary system is also advancing for spotting means of higher performance.

On one hand, the glass to be used as a substrate has a smaller effective fixed area for a probe and less charge than those of a membrane. Accordingly, the following various coating techniques are attempted for immobilization of DNA probes. Generally, poly-L lysine or silane is used in coatings. Further, the terminals of DNA probes are aminated and crosslinked to silane glass.

The mainstream of gene expression analysis conducted at present by use of the DNA microarrays described above lies in a system of monitoring gene expression by a two-fluorescence labeling method. The principle thereof is to detect a difference between two samples. In this method, mRNAs obtained form two different samples are labeled with different labels respectively and then subjected to competitive hybridization on a DNA microarray. Thereafter, the fluorescences of the two samples obtained on the DNA microarray are measured and compared.

The sample to be analyzed by the DNA microarray described above is generally RNA such as mRNA or entire RNA extracted from tissues or cells. Accordingly, the fluorescence described above is derived from a label such as Cy3-dUTP and Cy5-dUTP to be labeled in synthesizing cDNA from RNA. Further, an oligo-dT primer or a random primer and a reverse transcriptase are often used for labeling in synthesizing cDNA.

When nucleic acid is analyzed with the DNA microarray described above, there is the following problem. Because plural kinds of DNA probes are immobilized on the same substrate, plural kinds of probes having different nucleotide sequences are generally present on the microarray. However, the reaction conditions suitable for all DNA probes contained therein are not necessarily the same. Accordingly, it is difficult in the conventional DNA array to efficiently utilize all probes immobilized thereon, and it cannot be said that all desired targets are efficiently detected therewith. Accordingly, the result of detection with the DNA microarray is conventionally poor in reproducibility.

### BRIEF SUMMARY OF THE INVENTION

Under the circumstances described above, an object of the invention is to provide a probe-immobilized reaction array excellent in reaction efficiency and reproducibility.

As a result of extensive study, the present inventors found that the problem can be solved and the object can be achieved by the following means: that is, a probe-immobilized reaction array, comprising:

a substrate;

1st to nth reaction portions comprising bores formed independently in the inside of the substrate wherein n is an integer of 2 or more;

2 openings open from each of the 1st to nth reaction portions to a first face of the substrate; and

a probe group consisting of a 1st probe group immobilized on at least one face of the first reaction portion to an nth probe group immobilized on at least one face of the nth reaction portion wherein n is an integer of 2 or more,

wherein the first to nth probe groups comprise plural kinds of probes respectively, and the optimum reaction conditions of the probes are complete for each of the reaction portions.

According to such a structure, one optimum reaction condition can be applied to each reaction portion, i.e., to each probe group, and thus the best reaction result can be obtained for any probe groups. Accordingly, the probe-immobilized array can significantly improve not only reaction efficiency but also reproducibility.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiment of the invention, and together with the general description given above and the detailed description of the preferred embodiment given below, serve to explain the principles of the invention.

FIGS. 1A and 1B show one example of a probe-immobilized reaction array 1 according to one embodiment of the invention;

FIG. 2 is an illustration of a treatment system provided with the probe-immobilized reaction array according to the embodiment of the invention;

FIGS. 3A and 3B show one example of a probe-immobilized reaction array 1 according to one embodiment of the invention;

FIGS. 4A and 4B show one example of the probe-immobilized reaction array 1 according to the embodiment of the invention;

FIG. 5 shows an example of the temperature regulation of a probe-immobilized reaction array provided with a plurality of capillaries according to one embodiment of the invention;

FIG. 6 shows an analysis system provided with a probe-immobilized reaction array according to one embodiment of the invention;

FIG. 7 shows a mask pattern of one example of the probe-immobilized reaction array according to the embodiment of the invention;

FIG. 8 shows an example of a process for producing the probe-immobilized reaction array according to the embodiments of the invention;

FIG. 9 shows the concept of a tuple method;

FIG. 10 is a flowchart showing an example of a calculation filter; and

FIG. 11 shows one example of the probe-immobilized reaction array according to the embodiment of the invention.

### DETAILED DESCRIPTION OF THE INVENTION

The embodiments of the invention are described by reference to the following examples.

First Embodiment

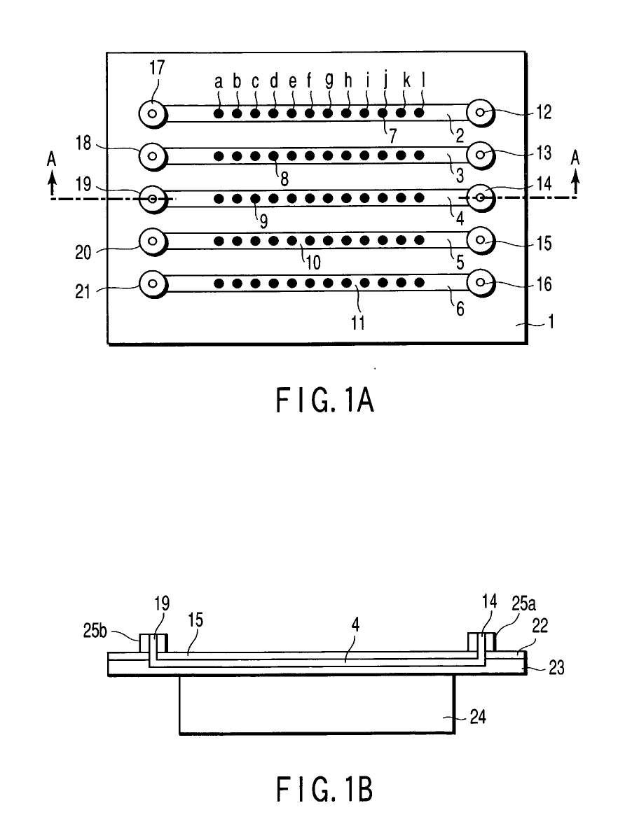

A first embodiment of the invention is shown in FIG. 1. FIG. 1A is a top plan view of a probe-immobilized reaction array 1 in this embodiment. The probe-immobilized reaction array 1 is an example of a probe-immobilized reaction array produced from a transparent glass substrate 22 and a silicon substrate 23. FIG. 1A shows each capillary containing a reaction portion formed on the silicon substrate 23 and nucleic acid probes immobilized thereon. FIG. 1B is a sectional view taken along the line A-A in FIG. 1A, of the probe-immobilized reaction array 1 arranged on a heater 24.

In the inside of the substrate of the probe-immobilized reaction array 1, a plurality of capillaries 2, 3, 4, 5 and 6 have been formed. Because the respective capillaries have been independently formed, a fluid contained in one capillary is not mixed with a fluid in another capillary. Further, each capillary is provided at both ends with openings. Specifically, capillaries 2, 3, 4, 5 and 6 have been provided with sample inlets 12, 13, 14, 15 and 16 and sample outlets 17, 18, 19, 20 and 21, respectively. The nucleic acid group 7 (a to l), nucleic acid group 8 (a to l), nucleic acid group 9 (a to l), nucleic acid group 10 (a to l) and nucleic acid group 11 (a to l) grouped according to their difference in Tm, each group having 12 spots in total, have been immobilized in capillaries 2, 3, 4, 5 and 6, respectively.

In the capillary 2, nucleic acid probes having 12 different nucleotide sequences, consisting of nucleic acid probes 7(a) to 7(l), have been immobilized. Each of the nucleic acid probes 7(a) to 7(l) has a plurality of identical Tm immobilized thereon. The Tm of the nucleic acid probe group immobilized in the capillary 2 is designated Tm1.

In the capillary 3, nucleic acid probes having 12 different nucleotide sequences, consisting of nucleic acid probes 8(a) to 8(l), have been immobilized. Each of the nucleic acid probes 8(a) to 8(l) has a plurality of identical Tm immobilized thereon. The Tm of the nucleic acid probe group immobilized in the capillary 3 is designated Tm2.

In the capillary 4, nucleic acid probes having 12 different nucleotide sequences, consisting of nucleic acid probes 9(a) to 9(l), have been immobilized. Each of the nucleic acid probes 9(a) to 9(l) has a plurality of identical Tm immobilized thereon. The Tm of the nucleic acid probe group immobilized in the capillary 4 is designated Tm3.

In the capillary 5, nucleic acid probes having 12 different nucleotide sequences, consisting of nucleic acid probes 10(a) to 10(l) have been immobilized. Each of the nucleic acid probes 10(a) to 10(l) has a plurality of identical Tm immobilized thereon. The Tm of the nucleic acid probe group immobilized in the capillary 5 is designated Tm4.

In the capillary 6, nucleic acid probes having 12 different nucleotide sequences, consisting of nucleic acid probes 11(a) to 11(l), have been immobilized. Each of the nucleic acid probes 11(a) to 11(l) has a plurality of identical Tm immobilized thereon. The Tm of the nucleic acid probe group immobilized in the capillary 6 is designated Tm5.

The difference in Tm among plural kinds of nucleic acids contained in each nucleic acid group may be within about ±2° C., preferably within about ±1°C.

Further, the difference in Tm between nucleic acid probes, that is, between Tm1 and Tm2, Tm2 and Tm3, Tm3 and Tm4, or Tm4 and Tm5 is preferably from about 2° C. to 30° C.

As used herein, the term “nucleic acid” refers to naturally occurring DNA and RNA as well as artificially synthesized nucleic acid analogues such as peptide nucleic acid, morpholino nucleic acid, methyl phosphonate nucleic acid and S-oligo-nucleic acid.

As used herein, the term “target nucleic acid” refers to nucleic acid to be detected by the nucleic acid probe. Generally, the nucleic acid probe is designed to have a nucleotide sequence complementary to its target nucleic acid. When a nucleic acid contained in a test sample has a nucleotide sequence possessed by the target nucleotide, there occurs hybridization between the nucleic acid probe and the target sequence. Accordingly, the nucleic acid contained in the test sample can be analyzed by detecting this hybridization. Detection of the hybridization may be conducted by a means known per se. A nucleotide sequence targeted in the target nucleic acid is called “target sequence”.

As used herein, the term “individuals” refers to arbitrary mammals including humans, dogs, cats, cattle, goats, pigs, sheep and monkey, as well as living things such as plants and insects other than mammals.

As used herein, the term “test sample” refers to biological samples such as cells, tissues, organs, blood, serum, lymph fluid, tissues, hair and earwax collected and prepared as necessary from individual living things, or to samples containing artificially synthesized or produced materials to be examined. The “test sample” may be a sample obtained by arbitrary or necessary pretreatment such as homogenization and extraction of biological samples. Depending on the intended biological sample, such pretreatment could be selected by those skilled in the art.

The probe-immobilized reaction array 1 described above can be produced, for example, in the following manner. Glass and silicon substrates having the same size are prepared and designated substrates 22 and 23, respectively. The substrate 23 is etched to form grooves. Desired nucleic acid probes have previously been grouped according to their difference in Tm. These nucleic acid probes in each group are immobilized by spotting onto the bottom of the groove. On one hand, the substrate 22 is provided with a through hole at a position corresponding to both ends of the groove of the substrate 23. Then, the substrate 22 is bonded to the substrate 23. By bonding a glass tube of desired length to each opening, connecting portions 25a and 25b are formed.

In the example described above, the substrate used as a lid makes use of a glass substrate, while the substrate for forming grooves makes use of a silicon substrate, but the material of the substrate is not particularly limited, so a silicon substrate may be used as the lid substrate, while a glass substrate may be used as the substrate for forming grooves. Alternatively, the two substrates may be made of the same material. Further, the member used may be determined such that a transparent member is arranged in the direction of observation. Alternatively, a substrate formed from plastic resin or rubber may be used. Further, substrates made of these materials or materials such as glass, silicon, plastic resin and rubber may be used in combination.

In the example described above, a plate substrate is used as the substrate, but the shape of the substrate is not limited.

A glass substrate or a silicon substrate can be provided with grooves and through holes by, e.g., microfabrication technology. A substrate of plastic resin and rubber can be provided with grooves and through holes by machining or molding. Means for immobilizing nucleic acid probes may be any means known in the art, for example by spotting or photochemical-immobilization. In the example described above, the nucleic acid probes are immobilized at the bottom of the groove of the substrate 23, but the probes may be immobilized on the substrate 22 or on a side of each reaction portion.

The probe-immobilized reaction array 1 provided with five capillaries is shown in the above example, but the number of arranged capillaries may be not less than or not more than five without any particular limitation. In the example described above, the respective groups, each consisting of nucleic acid groups classified depending on their difference in Tm, are immobilized onto different capillaries. However, nucleic acid probes to be contained in the same Tm group may be immobilized on two or more capillaries. Further, it is not always necessary that probes contained in a plurality of spots arranged on one capillary be of different types in each spot. That is, probes of the same type may be contained in two or more spots.

FIG. 1 shows an example where 12 spots are present in one capillary. However, the number of spots immobilized on one capillary is not particularly limited, so the number of spots may be one or more. Further, nucleic acid probes having the same optimum reaction condition may be immobilized in an arbitrary number of spots on one capillary, that is, the number of spots may be varied among the respective capillaries. All the capillaries contained in the probe-immobilized may not contain plural kinds of probes, or capillaries with no probe immobilized may be present.

Bonding of the two substrates may be conducted by means known per se before or after immobilization of nucleic acid probes. For example, silicon-quartz glass may be bonded by screen printing. For example, silicon-Pyrex glass may be bonded at high temperature and high voltage by anodic bonding.

The probe-immobilized reaction array 1 thus formed is subjected to hybridization at a suitable temperature for the Tm of probes arranged on each capillary. For example, the optimum reaction condition may be attained by controlling the temperature of each capillary, or by regulating the ion concentration and salt concentration while keeping all capillaries at a constant temperature.

For example, when the respective optimum temperatures for all capillaries having different optimum reaction temperatures are to be maintained by using only one heater 24, the concentration of a salt contained in the reaction solution used for hybridization may be regulated depending to the Tm value.

According to this embodiment, even in the case where Tm1 (optimum reaction temperature) is about 45° C., Tm2 is 50° C., Tm3 is 55° C., Tm4 is 60° C. and Tm5 is 65° C., the reaction of the nucleic acid probe with a target nucleic acid to be detected by the nucleic acid probe can be well conducted even if the temperature of all capillary reaction portions in the probe-immobilized reaction array 1 is kept at 45° C. That is, the optimum concentration of Tm2 to Tm5 can be achieved for example by regulating the concentration of a salt in the buffer used as a solution for a test sample containing the target nucleic acid.

For example, when a DNA probe having a Tm of 80° C. at a salt concentration of 1 M is used, the temperature of the reaction portion therefor is lowered by about 10° C. by reducing the salt concentration to 0.2 M while maintaining 80° C. Accordingly, in the case of heating at a constant temperature of 45° C. by heater 24, the concentration of a salt in the buffer should be regulated such that Tm is 65° C., because the reaction is often conducted at a temperature lower by about 20° C. than Tm. Further, when a DNA denaturant such as formamide is contained as a component in the solution used in actual hybridization reaction, the salt concentration should be further regulated as necessary.

The washing after the hybridization reaction is also important for realizing the accurate reaction. In the DNA hybridization reaction, mismatch should be reduced as much as possible, and the efficiency of hybridization with the target nucleic acid should be raised as much as possible. For this purpose, only a nucleic acid bound by mismatch to the nucleic acid probe should be removed while the normally hybridized target nucleic acid is not removed. The number of hydrogen bonds in a double-stranded chain formed from the nucleic acid probe and the mismatched nucleic acid in question is lower when the nucleic acids are mismatched than when the nucleic acids are matched. Accordingly, the Tm of the mismatched nucleic acids is lower. Accordingly, it is expected that the mismatched nucleic acid can be specifically removed by regulating the temperature or the salt concentration of the washing solution. The Tm of the mismatched nucleic acid is varied depending on the position and number of mismatches, thus making comprehensive definition of Tm difficult. However, the optimum conditions for desired analysis can be established by conducting an experiment for each group of capillaries. A method of preventing such mismatch also falls under the scope of the invention.

By utilizing the probe-immobilized reaction array 1 according to the embodiment of the invention, the conditions for hybridization reaction and the conditions for washing can be established separately for each capillary or for each unit consisting of a plurality of capillaries. Accordingly, the target sequence can be detected highly accurately with high reproducibility according to the embodiment of the invention. Further, mismatch in hybridization can also be prevented.

In this embodiment, the reaction portion contained in the probe-immobilized reaction array 1 is in the form of a capillary, but the shape is not particularly limited. That is, various device structures can be used wherein reaction portions separated physically from one another are formed, each of the reaction portions being provided with one or more openings that can be used as sample inlets and/or sample outlets. In the example described above, the number of reaction portions contained in the probe-immobilized reaction array 1 is five, but two or more reaction portions may be arranged without limitation to this number.

The reaction portions arranged in the probe-immobilized reaction array according to the invention refer to a region where the reaction (e.g., chemical or biochemical reaction) and treatment intended by the user. Accordingly, the reaction portions illustrated above are formed into capillaries, but the shape of the reaction portions is not particularly limited. The reaction portions may be formed independently, and for example, a lid or bottom having openings is arranged on a region partitioned by concave or convex portions, to form reaction portions each having an effective volume. The volume of each reaction portion in the reaction array of the invention may be 0.01 μl to 1 mL.

Further, according to the embodiment of this invention, not only the reaction array in a closed system wherein reaction portions each consisting of a bore have been formed in the substrate described above, but also a vessel divided by convex or concave portions or an undivided planar region can also be used in the embodiment of the invention insofar as the reaction portions are independently arranged by separating them from one another or by arranging a plurality of vertical holes to prevent diffusion of fluid. Such a unit and the reaction method of using the unit fall under the scope of the invention.

The reaction portion may be in the form of a well having a cubic, circular or elliptical bottom. The area of the bottom of the reaction portion may be the same as or different from the area of the ceiling of the reaction portion. As used herein, the “form of a well” means that the bottom or ceiling of the reaction portion includes not only a form having width in only a specific direction such as in a capillary, but also a form having width in any two-dimensional directions constituting the bottom or ceiling.

The “probe-immobilized reaction array” according to the invention may be a unit wherein a plurality of reaction portions having probes immobilized thereon as described above have been arranged in a necessary pattern on the same substrate. The probe-immobilized array can allow the probe to react with a target substance having high affinity for the probe. Preferably, the bonding formed by this reaction can be observed while it is contained in the substrate.

Strictly speaking, the reaction conditions for hybridization of nucleic acids are affected by both the nucleotide sequence of double-stranded nucleic acid formed during reaction and the salt concentration of the solution. The temperature at which double-stranded nucleic acid is transformed into single strands is referred to as Tm (melting temperature) and determined roughly by the number of hydrogen bonds and the environmental ion concentration. For constituent bases of nucleic acid, there are three hydrogen bonds in a GC bond and two hydrogen bonds in an AT bond. It follows that given nucleic acid of the same length, Tm is varied depending on its GC content. Tm is also increased with an increasing salt concentration in the solution. For example, when the salt concentration is changed 10 times, Tm is changed by several tens ° C.

According to the embodiment of the invention, the conventional problems in hybridization can be solved. Further, according to the idea of utilizing such characters regarded problematic in the prior art, there can be provided a probe-immobilized reaction array excellent in reaction efficiency and reproducibility as well as a reaction method of using the same.

The nucleic acid-immobilized reaction array having nucleic acid probes immobilized on a substrate is shown in this embodiment, but the nucleic acid probes may be replaced by protein, peptide, antigen or antibody probes or a combination thereof. In this case, a substance to bind specifically to the target substance to be detected may be bound as the probe onto the substrate.

In this embodiment, the Tm value and salt concentration were mentioned as factors for optimum reaction conditions. However, the factors for optimum reaction conditions are not particularly limited. For example, the reaction time may be selected as another factor for optimum reaction conditions. Further, these plural factors for optimum reaction conditions may be combined.

In this embodiment, a heater was arranged as a separate device on the reaction array in accordance with the invention, but a heater, a sensor and necessary wiring may be integrated into the reaction array.

In recent years, some protein chips having a plurality of proteins and peptides immobilized on a substrate have been disclosed. For example, chips having 10,000 or more proteins immobilized by a spotter for DNA microarray are disclosed in Science (2000) Sep 8; 289 (5485): 1673. Further, Ciphergen Ltd. proposes structural analysis of protein by a combination of a protein chip and a mass spectrometer, as well as a system for analysis of interaction therebetween. The embodiment of the invention can also be applied to the conventional protein chips known per se.

According to another aspect of the invention described above, there can also be provided a method of obtaining information on a target substance in an individual, which comprises using the probe-immobilized array of the invention. This method can be carried out for example as follows. First, test samples are obtained from individuals in a method known per se. Then, the test samples are added to a desired number of reaction portions arranged in the probe-immobilized reaction array according to the invention or to a desired number of probe-immobilized reaction arrays. The reaction conditions of each of the probe groups arranged in each reaction portion containing the test sample are regulated to achieve the maximum reaction conditions under which the probes are reacted with the target substance. Then, bonding of the probe to the target substance is detected whereby the presence of the target substance in the test sample is detected. This detection can be conducted by means known per se, depending on the target substance used. Then, the result obtained in this detection is used to obtain information on the target substance in the individuals.

The information obtained by this method includes, but is not limited to, information on gene expression, information on genome, information on protein expression, immunological information on antigen and antibody, and infections and diseases in the individuals as the subject. The probes for obtaining desired information may previously have been designed or selected.

Second Embodiment

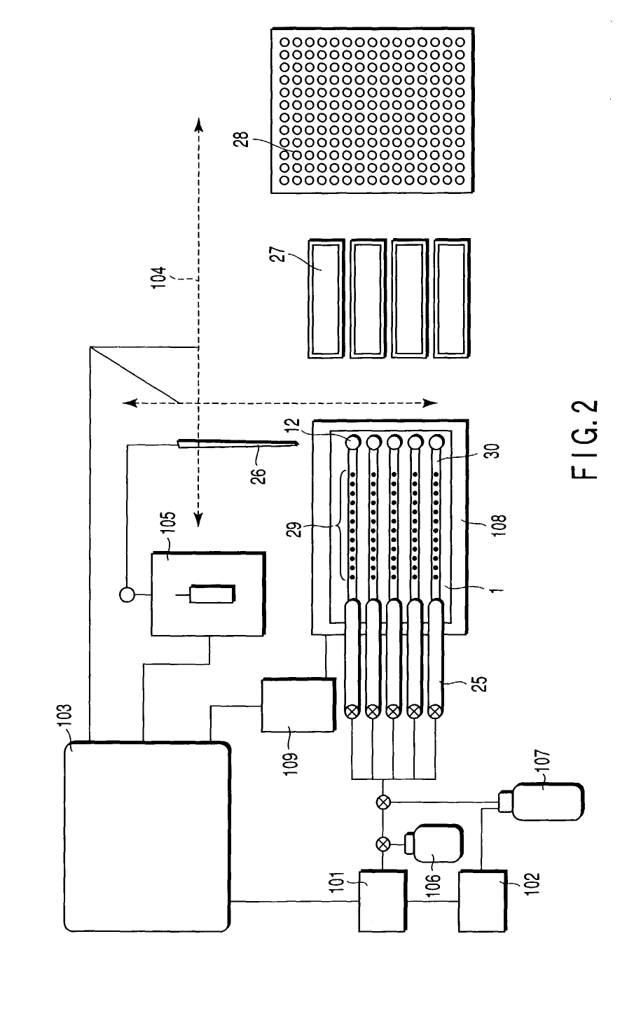

FIG. 2 shows an outline of a treatment system using the probe-immobilized reaction array 1 shown in the first embodiment. According to the invention, the optimum reaction conditions should be managed for each group of nucleic acid probes. As a result, the procedures can be troublesome. However, the treatment can be carried out efficiently by using the treatment system shown in this embodiment.

Now, one example of the treatment system of the invention is described by reference to FIG. 2. A discharge tube 25 is connected to a capillary 30 in the probe-immobilized reaction array 1. The discharge tubes are connected independently to each of the capillaries. Each tube is further connected to a suction pump. By the suction pump 101 and 102, a sample and reagents after use are discharged into a waste tank 106 and 107. A nozzle 26 is used to introduce fluid via each opening into each reaction portion in the probe-immobilized reaction array 1. The nozzle 26 is connected to a pipetting unit 105 and a X-Y-Z-axis driving unit 104. Desired pipetting and movement between the coordinates in this processing system are thereby made feasible. A plurality of reservoirs 27 are provided to accommodate washing buffers at various salt concentrations. Further reservoirs for accommodating reagents depending on the necessity may be arranged. Each well of a microtiter plate 28 accommodates a test sample. A heater 108 is arranged at the bottom of the probe-immobilized reaction array 1, and the temperature of the heater 108 is controlled by a temperature-controlling unit 109 including a sensor. Further, the temperature regulating unit 109, the suction pumps 101 and 102, the pipetting unit 105 and the X-Y-Z-axis driving unit 104 are controlled by the control unit 103.

Now, the procedures of conducting reaction in this embodiment are described. First, the capillaries 30 contained in the probe-immobilized reaction array 1 are described. All capillaries contained in the probe-immobilized reaction array 1 can be treated in the same manner. The capillaries 30 are previously grouped according to Tm, and each is provided with an immobilized nucleic acid probe group 29. A sample prepared at a suitable salt concentration for the optimum reaction conditions for the probe contained in the capillary 30 is introduced from the pipetting nozzle 26 through the sample inlet 12 into the reaction portion. After the reaction is conducted for a predetermined time at a desired temperature, a washing buffer is sucked from the reagent reservoir 27 by the pipetting nozzle 26 and then added to the capillary 30. The pipetting nozzle 26 is connected and regulated so as to enable transfer and injection of a reagent in a desired amount from the reagent reservoir 27 to the capillary 30. The thus constituted treatment system in accordance with this embodiment can omit the procedure of preparing the reagent and washing solution to attain a salt concentration suitable for the optimum reaction conditions for each probe contained in all capillaries 30 in the probe-immobilized reaction array 1. Washing is conducted usually with two or three buffer solutions. The reaction can be carried out automatically and accurately with good reproducibility by mechanically and accurately regulating the salt concentration of the washing buffer, the washing time, and the number of times washing is conducted.

This treatment system is used in the reaction using the probe-immobilized reaction array 1, whereby the reaction in the probe-immobilized reaction array in accordance with this embodiment can be accurately carried out to achieve good reproducibility. Accordingly, the probe-immobilized reaction array can be utilized more effectively.

Third Embodiment

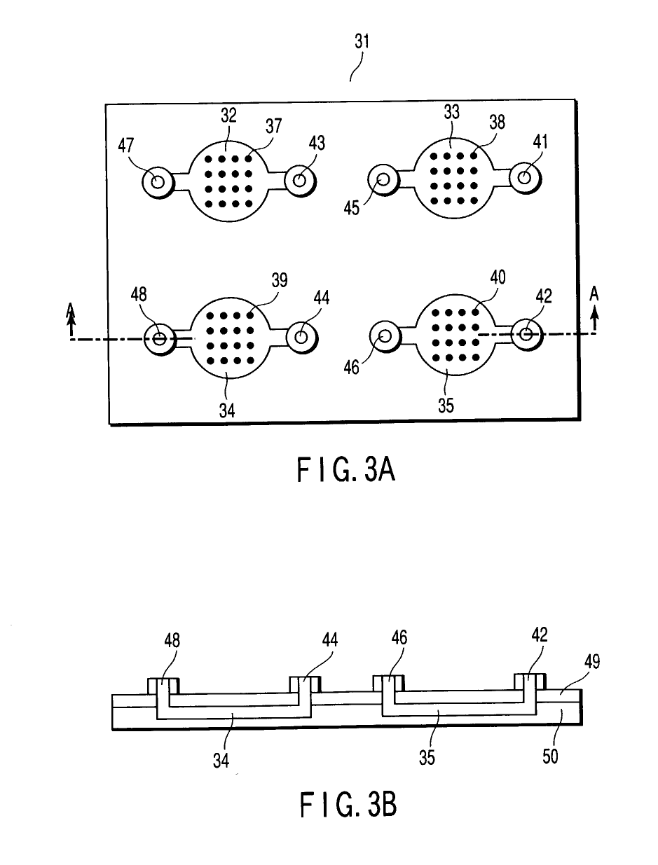

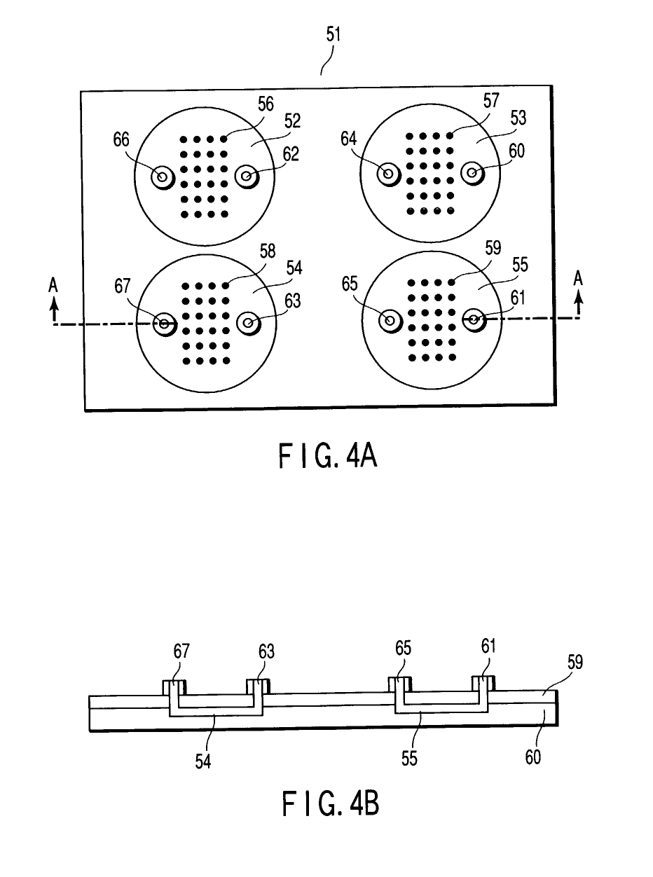

A probe-immobilized reaction array of the invention having a well-shaped reaction portion is shown in FIGS. 3 and 4.

FIG. 3A is a top plan view of a probe-immobilized reaction array 31 in accordance with this embodiment. A probe-immobilized reaction array 31 is an example of the probe-immobilized reaction array that, in the same manner as in the first embodiment, is produced from a transparent glass substrate 49 and a silicon substrate 50. In FIG. 3A, each of wells 32, 33, 34 and 35 and nucleic acid probes 37, 38, 39 and 40 formed on the silicon substrate 50 can be observed. FIG. 3B is a sectional view of the probe-immobilized reaction array 1 taken along the line A-A in FIG. 3A.

In the inside of the substrate for the probe-immobilized reaction array 31, the plurality of wells 32, 33, 34 and 35 are formed. Because the respective wells are independently formed, a fluid in one well will not be mixed with a fluid in another well. Further, each well has formed openings at both ends thereof. Specifically, the wells 32, 33, 34 and 35 have formed sample inlets 41, 42, 43 and 44 and sample outlets 45, 46, 47 and 48, respectively. The nucleic acid group 37 (a to p), nucleic acid group 38 (a to p), nucleic acid group 39 (a to p) and nucleic acid group 40 (a to p) grouped according to their difference in Tm, each group having 16 spots in total, have been immobilized on the wells 32, 33, 34 and 35, respectively.

FIG. 4A is a top plan view of another probe-immobilized reaction array 51 in accordance with this embodiment. The probe-immobilized reaction array 51 is an example of the probe-immobilized reaction array that, in the same manner as in the first embodiment, is produced from a transparent glass substrate 59 and a silicon substrate 60. In FIG. 4A, each of wells 52, 53, 54 and 55 and nucleic acid probes 56, 57, 58 and 59 formed on the silicon substrate 60 can be observed. FIG. 4B is a sectional view of the probe-immobilized reaction array 51 taken along the line A-A in FIG. 4A.

In the inside of the substrate for the probe-immobilized reaction array 51, the plurality of wells 52, 53, 54 and 55 are formed. Because the respective wells are independently formed, a fluid in one well will not be mixed with a fluid in another well. Further, each well has formed openings at both ends thereof. Specifically, the wells 52, 53, 54 and 55 have formed sample inlets 60, 61, 62 and 63 and sample outlets 64, 65, 66 and 67 respectively. The nucleic acid groups 56 (a to x), 57 (a to x), 58 (a to x) and 59 (a to x) grouped according to their difference in Tm, each group having 24 spots in total, have been immobilized on the wells 52, 53, 54 and 55, respectively.

The probe-immobilized reaction array according to the invention can be produced, used and changed according to the first and second embodiments. Further, the probe-immobilized reaction array may be used according to other embodiments described later.

The well-shaped reaction portion shown in this embodiment has a circular bottom and ceiling, but the shape of the bottom and ceiling of the well-shaped reaction portion is not particularly limited, and any shapes enclosed by a curve and/or a straight line can be used.

Fourth Embodiment

Now, a method of attaining the optimum reaction conditions by establishing the reaction temperatures of the capillaries 2, 3, 4, 5 and 6 arranged in the probe-immobilized reaction array 1 in the first embodiment is described. For example, such control may be temperature control of each capillary by using a commercial heat block. Alternatively, e.g., the following heating method can be used.

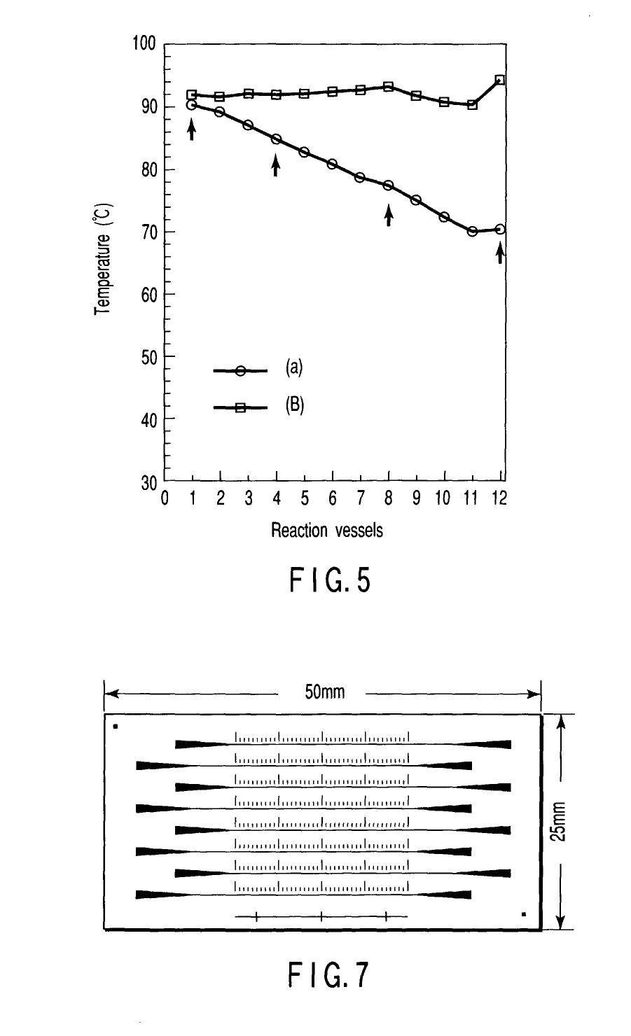

For example, using a probe-immobilized reaction array provided with 12 capillaries arranged side by side is described. Below each capillary, a resistance heating element and a temperature-sensing resistance are arranged in a pattern along the shape of the capillary. For convenience' sake, numbers 1, 2, 3 . . . 12 are given to capillaries in order from the end, among which resistance heating elements and temperature-sensing resistances in capillaries 1, 4, 8 and 12 are selected. The selected four capillaries are kept at 90° C., 81.8° C., 70.9° C., and 60° C., respectively, while the temperature of a solution in the reaction portion in each capillary is measured. The results are shown by (a) open circles in the graph in FIG. 5. Further, all the four capillaries are kept at 90° C., while the temperature of a solution in the reaction portion in each capillary is measured. The results are shown by (b) open squares in the graph in FIG. 5.

In the graph in FIG. 5, the capillary numbers are shown on the abscissa, while the temperatures of solutions contained in all the reaction portions in each capillary are shown on the ordinate. The arrows in the graph show the heated capillaries. As is evident from this graph, the temperatures of the reaction portions arranged in the 12 capillaries in one probe-immobilized reaction array are regulated continuously as a whole by regulating the temperature of the four capillaries. The temperature and temperature gradient of the reaction portions can be arbitrarily established depending on experimenter's need.

In the method described above, the temperatures of the reaction portions in the 12 capillaries were regulated by four temperature-regulating means. However, the number of temperature-regulating means may be not more than or not less than four, while the number of capillaries can also be varied as necessary.

In the foregoing description, the factor selected as the optimum reaction condition for each probe group was temperature. However, such factors selected as the optimum reaction conditions may be arbitrary selected from temperature, salt concentration and/or reaction time and may be independently regulated.

Fifth Embodiment

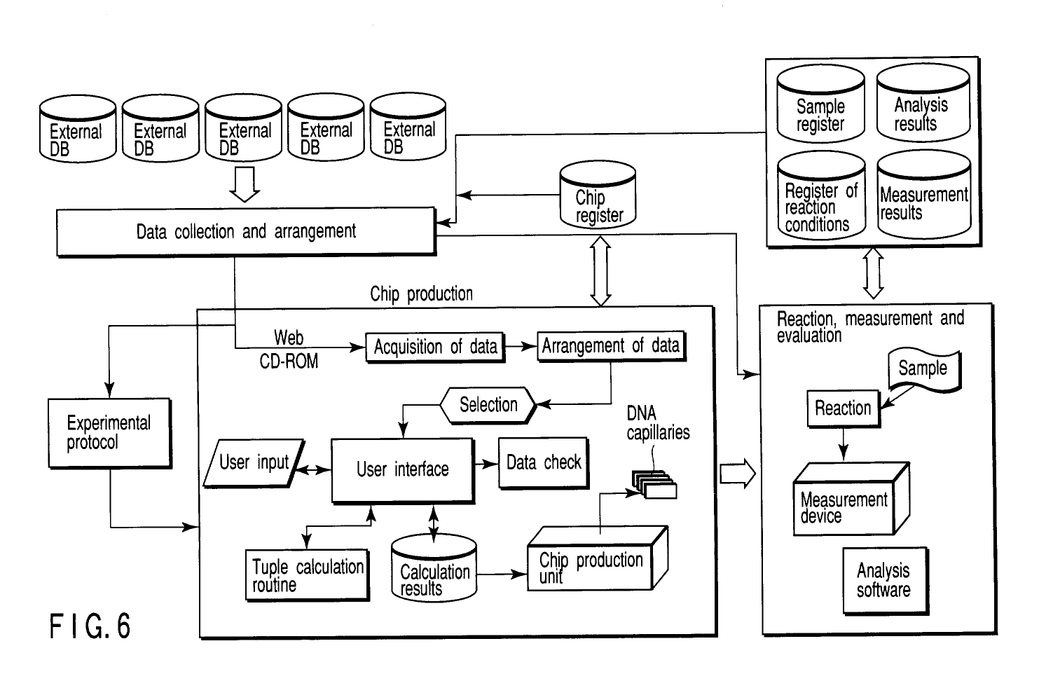

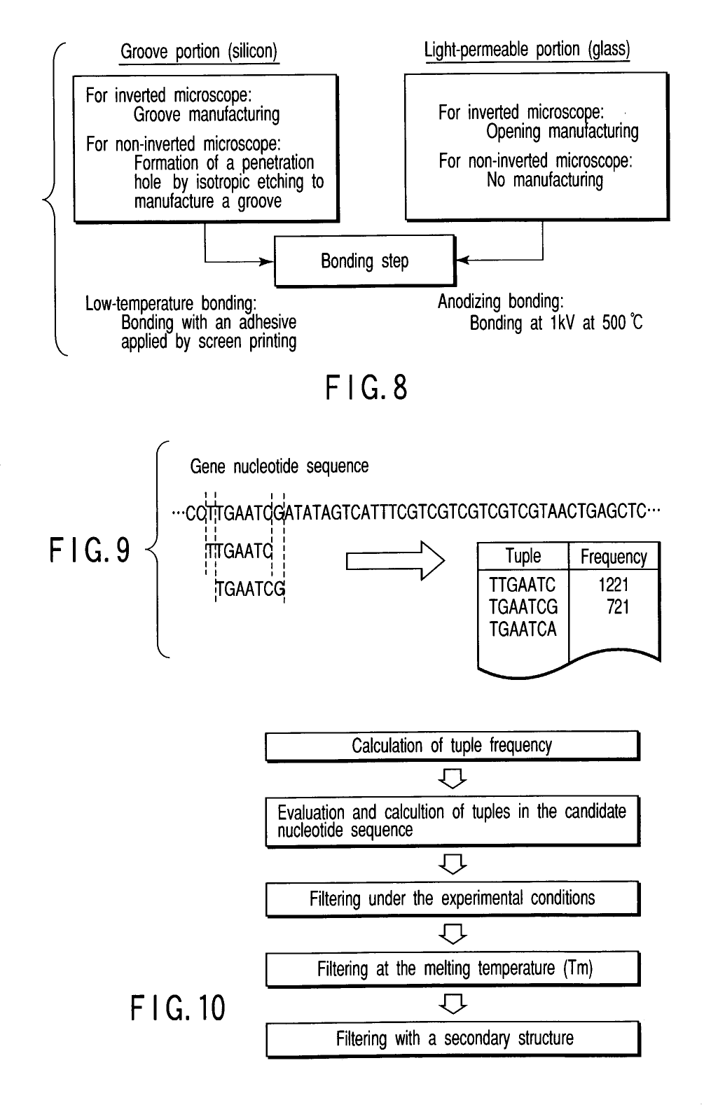

A further embodiment of the invention is described by reference to FIG. 6. This embodiment is related to an analysis system ranging from the design and production of the probe-immobilized reaction array according to the invention, to the acquisition of data based on assays conduced using the probe-immobilized reaction array.

This embodiment is a system wherein the reliability and accuracy of data are improved by (1) design of DNA probe groups grouped depending on Tm and production of reaction arrays having the designed DNA probes immobilized thereon according to the invention, (2) analysis using the probe-immobilized reaction arrays obtained in (1) above, (3) collection and arrangement of the analysis results obtained in item (2) above, or if necessary by repeating the cycle of (1) to (3).

This embodiment makes use of a unit (DNA capillary array) wherein the reaction portions in the probe-immobilized reaction array are shaped into capillaries, and DNA probes have been immobilized on the capillaries. However, the invention is not limited thereto. The analysis system shown in this embodiment includes the DNA capillary array, various devices, tools and units.

For designing DNA probes, a gene as the subject of analysis is determined, and a database is utilized for designing DNA probes specific to the gene. This database may be any database known in the art. The database is available from Web, CD-ROM, etc. However, a database available at present has noises and indefinite sequences. Accordingly, it cannot be utilized directly in designing the probe, and thus selection of reliable date is necessary. After the data selection, a design program is run to obtain an oligo-DNA probe corresponding to the objective gene. In this drawing, the design program indicates user's input, user interface, data check, tuple calculation routine and calculated results. In this design program, a tuple method is adopted for efficiently conducting designing, and candidate DNA probe sequences grouped depending on Tm are output via a specific filter, a secondary-structure filter and a Tm filter. DNA probes are synthesized on the basis of these results, and by using a DNA chip-producing unit, the DNA probes grouped depending on Tm are immobilized onto DNA capillaries, whereby the DNA capillary arrays in this embodiment can be produced. The information on this production may be used in reference to the chip register or in renewal of the register.

The reaction, measurement and evaluation using the DNA capillary arrays produced are carried out by a liquid-handling unit for conducting the reaction as well as a fluorescence-measuring system. This measuring portion is provided with optical measuring elements for irradiating the respective probes in desired order with a measurement beam or for receiving a fluorescence signal. Data on the measurement result measured in the measuring portion and data on the analysis result analyzed by analysis software, together with information on the reagent register for individuals and information on the reaction condition register for reaction conditions, are stored in the database as data to be used later in various kinds of analysis.

[Design of Oligo DNA Probe]

Now, a method of designing the nucleotide sequence of a nucleic acid probe (for example, an oligonucleotide probe etc.) to be used in the DNA capillary array is described.

The DNA probe used in the DNA capillary array includes oligonucleotide probes and cDNA probes. The oligonucleotide probe is superior to the cDNA probe in the following respects: (1) less error in cross-hybridization; (2) easier preparation of the probe; (3) rapider hybridization; and (4) capable of distinction between selective transcription and selective splicing. For design of the oligonucleotide probe, the nucleotide sequence of genome DNA should be sufficiently determined, and as the genome project is rapidly advancing, this condition comes to be satisfied for many living things.

In the conventional DNA chip, probes are accumulated on the two-dimensionally expanding surface of the substrate. These probes are subjected to the same hybridization conditions and washing conditions, so errors attributable to false positive or false negative reactions can occur unless all the probes have similar Tm. Further, the analysis is subject to the dynamic range of the detector used, thus making it difficult to accurately detect samples expressed at considerably different expression levels.

On the other hand, the DNA capillary array provided with a plurality of independent capillaries according to the invention allows washing conditions and conditions such as sample concentration to be adapted for each of the capillaries on the same DNA capillary array. To utilize this property, a tuple method that is a method of designing probe sequences grouped efficiently depending on Tm or expression frequency (N. Uchikoga, A. Suyama, Genome Informatics, 9, 388-389 (1998)) can also be used. An embodiment of using this method also falls under the scope of the invention.

The tuple method is a method of determining a nucleotide sequence used as a probe, on the basis of the result of counting the number of times a nucleotide sequence consisting of a few nucleotides called a tuple (e.g. 7-nucleotide sequence in this example) occurs in the entire gene. This counting may be conducted only once initially.

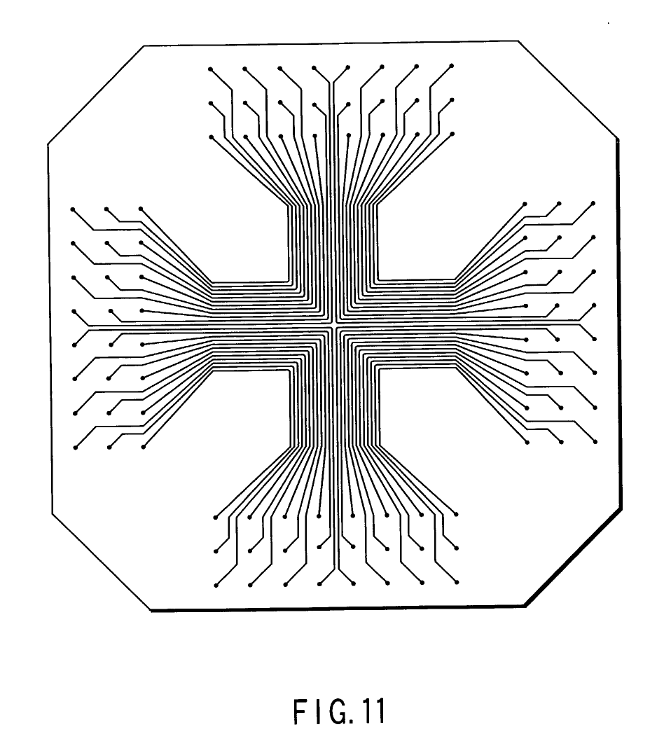

As shown in FIG. 9, whether a table occurring highly frequently is present or not is first determined by examining a tuple contained in a nucleotide sequence previously selected as a candidate. Thereafter, specificity is calculated as to whether the candidate tuple is a “ubiquitous” sequence in the entire gene. In the actual method, tuple frequency is first calculated, and then the candidate sequence is evaluated as a tuple, and a sequence having high specificity is selected as a tuple, and after application of limits determined by experimental conditions, the tuple is further screened with a filter allowing the candidate having thermal properties suitable for the experiment to pass therethrough. Because DNA has complementarily binding properties, sufficiently long single-stranded DNA can bind intramolecularly thus hardly undergoing hybridization reaction. Accordingly, whether or not the candidate can take such a structure is also calculated in order to exclude the candidate having such a structure. The procedures described above are shown in FIG. 10.

By these methods, the design of a DNA probe sequence consisting of 30 nucleotides was attempted in order to identify an entire ORF (open reading frame) region in sprouted yeast. Table 1 shows 6 to 10 candidates for a DNA probe to detect each of three kinds of genes as partial regions of the ORF. In this case, the Tm groups were four groups having 64±2° C., 60±2° C., 54±2° C. and 52±2° C., respectively.

TABLE 1
Results of calculation of DNA probe sequences

| | | | | | | | | |
|---|---|---|---|---|---|---|---|---|
| | | | | | The | The | | |
| Tin | Freq. | | 2ndary | Min | nearest | nearest | Probe | |
| group | Index | Tm/° C. | struct. | DH | ORF | position | position | Probe sequence |

sec: YAL068C
strong similarity to subtelomeric encoded proteins [SP: YGZF_YEAST]

| | | | | | | | | |
|---|---|---|---|---|---|---|---|---|
| 4 | −239.01 | 65.31 | −7.42 | 1 | 27 | 3 | 1523271 | ggtgtcgctgccatcgctgctactgcttct |
| 3 | −233.99 | 59.21 | −4.37 | 1 | 52 | 1 | 809064 | cttctgcaaccaccactctagctcaatctg |
| 3 | −234.66 | 61.76 | −6.17 | 1 | 139 | 3 | 1523383 | tagcccaatactacat ttccaa ccgccc |
| 4 | −234.99 | 62.94 | −6.72 | 1 | 140 | 3 | 1523384 | agcccaatactacatgttccaagccgccca |
| 2 | −238.02 | 56.65 | −8.07 | 1 | 128 | 3 | 1523372 | cagagctcacttagcccaatactacatgtt |
| 3 | −238.04 | 61.62 | −8.47 | 1 | 237 | 1 | 809249 | ttgaccggtattgctccagaccaagtgacc |

sce: YAL063C
FL09; FL01 homolog [SP:YAG3_YEAST]

| | | | | | | | | |
|---|---|---|---|---|---|---|---|---|
| 4 | −246.39 | 64.67 | −12.92 | 8 | 60 | 0 | 218179 | gttgtctctgcgactacagcggcatgcctg |
| 3 | −243.07 | 59.22 | −8.12 | 7 | 534 | 0 | 218651 | gaacaacctcccatcacgtcgactaacttc |
| 4 | −242.53 | 63.21 | −6.22 | 4 | 666 | 0 | 218783 | tcaaatgccgttgcctggggtacacttcca |
| 3 | −241.37 | 61.58 | −6.12 | 4 | 663 | 0 | 218780 | tactcaaatgccgttgcctggggtacactt |
| 4 | −242.62 | 63.09 | −13.77 | 3 | 2455 | 0 | 204223 | caaccgagccatggaccggtactttcacct |
| 3 | −243.03 | 61.17 | −7.82 | 2 | 2611 | 0 | 206677 | ccagctttatcacgtctgcgcgtccaatta |
| 3 | −245.32 | 61.43 | −14.27 | 2 | 2447 | 0 | 204215 | tacaactacaaccgagccatggaccggatc |
| 3 | −243.7 | 60.39 | −11.17 | 2 | 2882 | 0 | 206906 | ttctgagagcgaaacgggttcagctagttc |
| 4 | −244.3 | 63.62 | −7.82 | 2 | 2608 | 0 | 206674 | tcaccagctttatcacgtctgcgcgtccaa |
| 3 | −241.53 | 60.57 | −10.07 | 2 | 492 | 0 | 218609 | ctatcagtcggtggtagcattgcgttcgaa |

sce: YAR009C
unknown

| | | | | | | | | |
|---|---|---|---|---|---|---|---|---|
| 1 | −231.38 | 54.17 | −4.42 | 8 | 137 | 15 | 336582 | aatgcacagacaaattcgatactcacttaa |
| 2 | −231.12 | 56.47 | −4.97 | 8 | 135 | 13 | 220613 | ccaatgcacagacaaattcgatactcactt |
| 3 | −231.52 | 60.8 | −8.42 | 7 | 128 | 1 | 261633 | gcgcatgccaatgcacagacaaattcgata |
| 2 | −237.32 | 56.68 | −8.12 | 1 | 2842 | 2 | 89281 | ttagagctccaggtcaaccaggtctttata |
| 3 | −238.67 | 61.57 | −11.12 | 1 | 413 | 1 | 223383 | gcgttgggtttatccattacacgaccgtcg |
| 2 | −232.62 | 57.19 | −9.07 | 1 | 408 | 1 | 223378 | aaattgcgttgggtttatccattacacgac |

[Liquid-Handling Unit]

In introduction of a sample into the DNA capillary array, reaction control and washing, a very small amount of a solution is required, and thus a special system has been developed. The liquid-handling unit is to feed various liquids to the DNA capillary array. In particular, accurate feeding of liquids accurately to a large number of capillaries requires regulation of liquid flow for each capillary, and accurate regulation of liquids to capillaries also requires valves whose number corresponds to the number of capillaries. For downsizing and higher accuracy of the unit as a whole, valve arrays are mounted by microprocessing technology, and the ineffective volume of the connector is reduced most desirably to the minimum for highly accurate regulation of liquid. However, because development of valve arrays is expected to be considerably difficult at present, valve arrays are realized using a plurality of commercial small valves.

Valve arrays to be preferably used according to the embodiments of the invention include e.g. those proposed by Shuichi Shoji, The Japan Society of Mechanical Engineers, Robotics and Mechanics Lecture (1998)).

Although the DNA capillary array as one example of this embodiment described above is a prototype, a unit adapted therefor to enable treatment of eight capillaries was developed. This unit is constituted to regulate the temperature of only the capillary portion having DNA probes immobilized thereon and to enable washing and introduction of about 5 μL sample solution per capillary. These treatments can be completely automated to realize reproducibility.

[High Density of Capillaries]

The probe-immobilized immobilized reaction array used according to the embodiments of the invention may be provided with capillaries in the pattern shown in FIG. 11. That is, the capillaries according to the invention may be bent partially or as a whole.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A probe-immobilized reaction array, comprising:
a substrate;
1st to nth reaction portions comprising bores formed independently in the inside of the substrate wherein n is an integer of 2 or more;
2 openings open from each of the 1st to nth reaction portions to a first face of the substrate; and
a probe group consisting of a first probe group immobilized on at least one face of the first reaction portion to an nth probe group immobilized on at least one face of the nth reaction portion wherein n is an integer of 2 or more,
wherein the first to nth probe groups comprise plural kinds of probes, respectively, and the optimum reaction conditions of the probes are complete for each of the reaction portions.

2. A probe-immobilized reaction array according to claim 1, wherein at least two groups out of the 1st to nth probe groups (n: an integer of 2 or more) have the optimum reaction conditions different from each other.

3. A probe-immobilized reaction array according to claim 1, wherein the reaction portion is in the form of a capillary.

4. A probe-immobilized reaction array according to claim 3, wherein the opening is open from both ends in the longitudinal direction of the capillary-shaped reaction portion to the first face of the substrate.

5. A probe-immobilized reaction array according to claim 1, wherein the reaction portion is in the form of a well.

6. A probe-immobilized reaction array according to claim 1, wherein the probe is nucleic acid.

7. A probe-immobilized reaction array according to claim 6, wherein the complete optimum reaction conditions are that Tm is similar.

8. A probe-immobilized reaction array according to claim 7, wherein the difference in Tm among all probes arranged in the 1st reaction portion is ±2° C.

9. A probe-immobilized reaction array according to claim 7, wherein the difference in Tm among all probes arranged in the 1st reaction portion is ±1° C.

10. A probe-immobilized reaction array according to claim 1, wherein the probe is selected from the group consisting of a peptide, a protein, an antigen and an antibody.

11. A probe-immobilized reaction array according to claim 10, wherein the complete optimum reaction conditions are that all probes arranged in one reaction portion have similar optimum pH and salt concentration for maintaining the activity of probes arranged in one reaction portion.

12. A reaction method of using the probe-immobilized reaction array described in claim 1, comprising:
(1) adding test samples to the 1st to nth reaction portions arranged in the probe-immobilized reaction array described in claim 1; and
(2) reacting the probe groups with the target substance while the reaction conditions in the 1st to nth reaction portions are regulated depending on the optimum reaction conditions in each reaction portion.

13. A method according to claim 12, wherein the reaction conditions are regulated by using a composition for achieving the optimum reaction conditions for each probe group, as the composition of a solution present in the reaction portions in carrying out the reaction (2) above.

14. A method according to claim 12, wherein the regulation of the reaction conditions is conducted by selecting at least one means selected from the group consisting of regulation of the temperature and the salt concentration in the reaction portions in conducting the reaction in (2) above, regulation of the reaction time in conducting the reaction in (2) above, and regulation of a combination of the above.

15. A method according to claim 12, wherein the reaction in (2) above is followed by washing each of the reaction portions with a solution having a composition for satisfying the optimum reaction conditions for each probe group.

16. A method of detecting a target substance with the probe-immobilized reaction array described in claim 1, comprising:
(1) adding test samples to the 1st to nth reaction portions arranged in the probe-immobilized reaction array described in claim 1;
(2) reacting the probe groups with the target substance while the reaction conditions in the 1st to nth reaction portions are regulated depending on the optimum reaction conditions in each reaction portion; and
(3) detecting the bonding of the probe to the target substance thereby detecting the presence of the target substance in the test sample.

17. A method according to claim 16, wherein the reaction in (2) above is followed by washing each of the reaction portions with a solution having a composition for satisfying the optimum reaction condition for the probe groups.

18. A method of obtaining information on a target substance in an individual by using the probe-immobilized reaction array of claim 1, comprising:
(1) obtaining test samples from individuals;
(2) adding the test samples to the 1st to nth reaction portions arranged in the probe-immobilized reaction array described in claim 1;
(3) reacting the probe groups with the target substance while the reaction conditions in the 1st to nth reaction portions are regulated depending on the optimum reaction conditions in each reaction portion;
(4) detecting the bonding of the probe to the target substance thereby detecting the presence of the target substance in the test sample; and
(5) obtaining information on the target substance in the individuals, from the results obtained by the detection described in (4) above.

19. A method of obtaining information on a target substance in an individual by using the probe-immobilized reaction array described in claim 1, comprising:
(1) obtaining test samples from individuals;
(2) adding the test samples obtained in (1) above to a plurality of reaction portions arranged in at least one probe-immobilized reaction array;
(3) reacting the probe groups with the target substance while the reaction conditions in the reaction portions in (2) above are regulated depending on the optimum reaction conditions in each reaction portion;
(4) detecting the bonding of the probe to the target substance thereby detecting the presence of the target substance in the test sample;
(5) analyzing and/or outputting the results obtained by the detection in (4) above while taking the respective reaction conditions into consideration; and
(6) analyzing and/or outputting the results in (5) above thereby obtaining information on the target substance in the individuals.

20. A method of obtaining information on a target substance in an individual by using the probe-immobilized reaction array described in claim 6, comprising:
(1) obtaining nucleic acid samples from individuals;
(2) labeling nucleic acids in the nucleic acid samples;
(3) adding the nucleic acid samples labeled in (2) above to the 1st to nth reaction portions arranged in the probe-immobilized reaction array described in claim 6;
(4) reacting the probe groups with the target substance while the reaction conditions in the 1st to nth reaction portions are regulated depending on the optimum reaction conditions in each reaction portion;
(5) detecting the bonding of the probe to the target substance thereby detecting the presence of the target substance in the test sample; and
(6) obtaining information on the gene in the individuals, from the results obtained by the detection in (5) above.
